(19) United States
(12) Patent Application Publication
(10) Pub. No.: US 2010/0267755 A1
(43) Pub. Date: Oct. 21, 2010

(54) PYRIMIDINE DERIVATIVES

(75) Inventors: Gianpaolo Bravi, Hertfordshire (GB); Malcolm Clive Carter, Hertfordshire (GB); Richard Howard Green, Hertfordshire (GB); Jennifer Margaret Doughty, Gorham, ME (US); Charles David Hartley, Hertfordshire (GB); Alan Naylor, Hertfordshire (GB); Martin Pass, Hertfordshire (GB); Jeremy John Payne, Hertfordshire (GB); Neil Anthony Pegg, Hertfordshire (GB)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignees: GLAXO GROUP LIMITED, Middlesex (GB); Jennifer Margaret GREEN, Hertfordshire (GB)

(21) Appl. No.: 10/477,546

(22) PCT Filed: May 23, 2002
(86) PCT No.: PCT/GB2002/002408

§ 371 Date: Jun. 23, 2006

(30) Foreign Application Priority Data
May 25, 2001 (GB) 0112803.2

(51) Int. Cl. A61K 31/505; A61P 29/00; C07D 239/42; C07D 403/12; C07D 405/12; C07D 409/12; A61K 31/506

(52) U.S. Cl. 514/275; 544/332; 544/331

(57) ABSTRACT

The invention provides the compounds of formula (I)

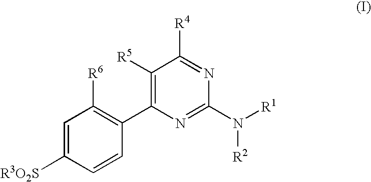

and pharmaceutically acceptable salts thereof, in which:
R¹ and R² are independently selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁷R⁸)ₙ and B(CR⁷R⁸)ₙ;
R³ is selected from the group consisting of C₁₋₆alkyl, NH₂ and R¹⁰CONH;
R⁴ is C₁₋₂alkyl substituted by one to five fluorine atoms;
R⁵ is selected from the group consisting of H, C₁₋₄alkyl, C₁₋₂alkyl substituted with one to five fluorine atoms, halogen and C₃₋₁₀cycloalkylC₀₋₆alkyl, with the proviso that when R⁶ is H R⁵ is not H.
R⁶ is selected from the group consisting of H, C₁₋₄alkyl, C₁₋₂alkyl substituted with one to five fluorine atoms, halogen, C₁₋₄alkoxy, CN, NO₂, C₁₋₆alkylOCO, NH₂CO, C₁₋₆alkylNHCO, NH₂, C₁₋₆alkylNH, (C₁₋₆alkyl)₂N, (C₁₋₆alkyl)₂NCO, C₁₋₆alkylCONH, NH₂SO₂, C₁₋₆alkylNHSO₂, (C₁₋₆alkyl)₂NSO₂, C₁₋₆alkylSO₂NH, ArSO₂NH, C₁₋₆alkylSO₂, ArSO₂, C₃₋₁₀cycloalkylC₀₋₆alkyl, C₃₋₆alkenyl and C₃₋₆alkynyl, with the proviso that when R⁵ is H R⁶ is not H.
R⁷ and R⁸ are independently selected from H or C₁₋₆alkyl;
A is an unsubstituted 5- or 6-membered heteroaryl or an unsubstituted 6-membered aryl, or a 5- or 6-membered heteroaryl or a 6-membered aryl substituted by one or more R⁹;
R⁹ is selected from the group consisting of hydroxy, halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
R¹⁰ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl;
B is selected from the group consisting of

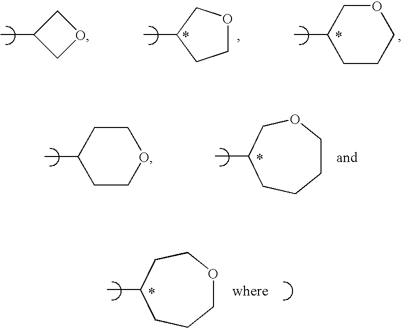

defines the point of attachment of the ring; and
n is 0 to 4.

Compounds of formula (I) are potent and selective inhibitors of COX-2 and are of use in the treatment of pain, fever and inflammation of a variety of conditions and diseases.

This invention relates to pyrimidine derivatives, to processes for their preparation, to pharmaceutical compositions containing them and to their use in medicine.

The enzyme cyclooxygenase (COX) has recently been discovered to exist in two isoforms, COX-1 and COX-2. COX-1 corresponds to the originally identified constitutive enzyme while COX-2 is rapidly and readily inducible by a number of agents including mitogens, endotoxin, hormones, cytokines and growth factors. Prostaglandins generated by the action of COX have both physiological and pathological roles. It is generally believed that COX-1 is largely responsible for the important physiological functions such as maintenance of gastrointestinal integrity and renal blood flow. In contrast the inducible form, COX-2, is believed to be largely responsible for the pathological effects of prostaglandins where rapid induction of the enzyme occurs in response to such agents as inflammatory agents; hormones, growth factors and cytokines. A selective inhibitor of COX-2 would therefore have anti-inflammatory, anti-pyretic and analgesic properties, without the potential side effects associated with inhibition of COX-1. We have now found a novel group of compounds which are both potent and selective inhibitors of COX-2.

The invention thus provides the compounds of formula (I)

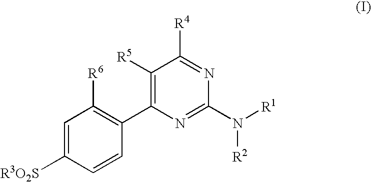

and pharmaceutically acceptable salts thereof, in which:

R¹ and R² are independently selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁷R⁸)ₙ and B(CR⁷R⁸)ₙ;

R³ is selected from the group consisting of C₁₋₆alkyl, NH₂ and R¹⁰CONH;

R⁴ is C₁₋₂alkyl substituted by one to five fluorine atoms;

R⁵ is selected from the group consisting of H, C₁₋₄alkyl, C₁₋₂alkyl substituted with one to five fluorine atoms, halogen and C₃₋₁₀cycloalkylC₀₋₆alkyl, with the proviso that when R⁶ is H R⁵ is not H.

R⁶ is selected from the group consisting of H, C₁₋₂alkyl substituted with one to five fluorine atoms, halogen, C₁₋₄alkoxy, CN, NO₂, C₁₋₆alkylOCO, NH₂CO, C₁₋₆alkylNHCO, NH₂, C₁₋₆alkylNH, (C₁₋₆alkyl)₂N, (C₁₋₆alkyl)₂NCO, C₁₋₆alkylCONH, NH₂SO₂, C₁₋₆alkylNHSO₂, (C₁₋₆alkyl)₂NSO₂, C₁₋₆alkylSO₂NH, ArSO₂NH, C₁₋₆alkylSO₂, ArSO₂, C₃₋₁₀cycloalkylC₀₋₆alkyl, C₃₋₆alkenyl and C₃₋₄alkynyl, with the proviso that when R⁵ is H R⁶ is not H.

R⁷ and R⁸ are independently selected from H or C₁₋₆alkyl;

A is an unsubstituted 5- or 6-membered heteroaryl or an unsubstituted 6-membered aryl, or a 5- or 6-membered heteroaryl or a 6-membered aryl substituted by one or more R⁹;

R⁹ is selected from the group consisting of hydroxy, halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;

R¹⁰ is selected from the group consisting of H, C₁₋₆alkoxy, C₁₋₆alkylOC₁₋₆alkyl, phenyl, HO₂CC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl;

B is selected from the group consisting of

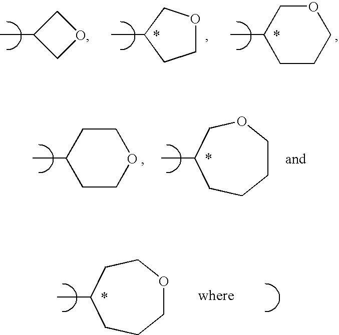

defines the point of attachment of the ring; and

n is 0 to 4.

Suitable pharmaceutically acceptable salts include acid addition salts formed with the amine functionality NR¹R². Pharmaceutically acceptable salts include those described by Berge, Bighley and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicyclic, methanesulfonic, p-toluenesulfonic, ethanedisulfonic, acetic, propionic, tartaric, salicyclic, citric, gluconic, aspartic, stearic, palmitic, itaconic, glycolic, p-aminobenzoic, glutamic, taurocholic, benzenesulfonic, hydrochloric, hydrobromic, sulfuric, cyclohexylsulfamic, phosphoric and nitric acids.

It will be appreciated that, for pharmaceutical use, the salts referred to above will be the physiologically acceptable salts, but other salts may find use, for example in the preparation of compounds of formula (I) and the physiologically acceptable salts thereof.

The term ‘halogen’ is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

The term 5-membered heteroaryl means a heteroaryl selected from the following:

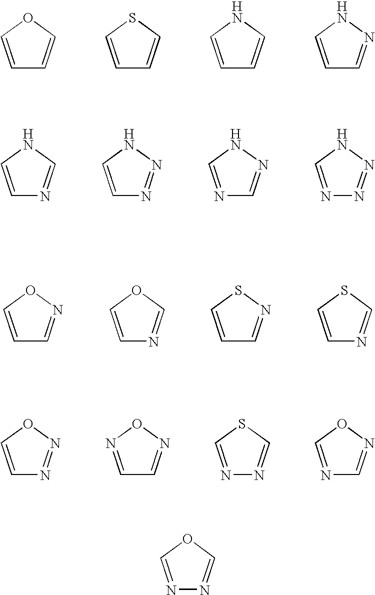

The term 6-membered heteroaryl means a heteroaryl selected from:

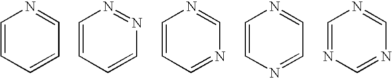

The term 6-membered aryl means:

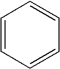

It is to be understood that the present invention encompasses all isomers of the compounds of formula (I) and their pharmaceutically acceptable salts, including all geometric, tautomeric and optical forms, and mixtures thereof (e.g. racemic mixtures). In particular when the ring B lacks a plane of symmetry the compounds of formula (I) contain a chiral centre as indicated therein by the asterisk *. Furthermore, it will be appreciated by those skilled in the art that when R⁴ and R⁵ in formula (I) are different the corresponding compounds contain at least one chiral centre, by virtue of the asymmetric carbon atom defined thereby, and that such compounds exist in the form of a pair of optical isomers (i.e. enantiomers).

In one aspect the invention provides compounds of formula (I) and pharmaceutically acceptable salts thereof, in which:

R¹ and R² are independently selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁷R⁸)ₙ and B(CR⁷R⁸)ₙ;

R³ is selected from the group consisting of C₁₋₆alkyl, NH₂ and R¹⁰CONH;

R⁴ is C₁₋₂alkyl substituted by one to five fluorine atoms;

R⁵ is selected from the group consisting of H, C₁₋₄alkyl, C₁₋₂alkyl substituted with one to five fluorine atoms, and C₃₋₁₀cycloalkylC₀₋₆alkyl, with the proviso that when R⁶ is H R⁵ is not H.

R⁶ is selected from the group consisting of H, C₁₋₄alkyl, C₁₋₂alkyl substituted with one to five fluorine atoms, halogen and C₁₋₄alkoxy, with the proviso that when R⁶ is H R⁶ is not H.

R⁷ and R⁸ are independently selected from H or C₁₋₆alkyl;

A is an unsubstituted 5- or 6-membered heteroaryl or an unsubstituted 6-membered aryl, or a 5- or 6-membered heteroaryl or a 6-membered aryl substituted by one or more R⁹;

R⁹ is selected from the group consisting of halogen, C₁₋₆alkyl, C₁₋₆alkyl substituted by one or more fluorine atoms, C₁₋₆alkoxy, C₁₋₆alkoxy substituted by one or more F, NH₂ SO₂ and C₁₋₆alkyl SO₂;

R¹⁰ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl;

B is selected from the group consisting of

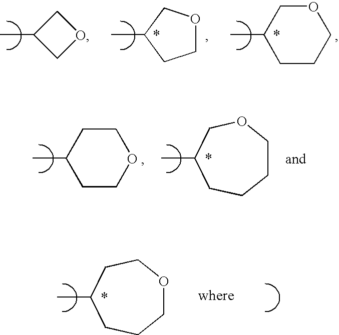

defines the point of attachment of the ring; and

n is 0 to 4.

In another aspect of the invention R¹ is H,

In another aspect of the invention R² is selected from the group consisting of C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl and A(CR⁷R⁸)ₙ.

In another aspect of the invention R² is selected from the group consisting of C₁₋₆alkyl, C₃₋₁₀cycloalkylC₀₋₆alkyl (such as C₃₋₁₀cycloalkyl or C₃₋₇cycloalkylmethyl), A(CR⁷R⁸)ₙ, and B(CR⁷R⁸)ₙ.

In another aspect of the invention R² is selected from the group consisting of C₁₋₆alkyl, C₃₋₁₀cycloalkylC₀₋₆alkyl (such as C₃₋₁₀cycloalkyl or C₃₋₇cycloalkylmethyl) and A(CR⁷R⁸)ₙ.

Representative examples of R² when it is C₁₋₆alkyl include n-propyl, n-butyl and s-butyl. Representative examples of R² when it is C₃₋₁₀cycloalkylC₀₋₆alkyl include cyclohexyl.

In another aspect of the invention R³ is C₁₋₆alkyl, such as C₁₋₃alkyl.

Representative examples of R³ include methyl.

In another aspect of the invention R⁴ is CHF₂, CH₂F or CF₃. In another aspect R⁴ is CF₃.

In another aspect of the invention R⁵ is H or C₁₋₄alkyl, with the proviso that when R⁶ is H R⁵ is not H.

Representative examples of R⁵ include H and methyl.

In another aspect of the invention R⁶ is selected from the group consisting of H, C₁₋₂alkyl (e.g. methyl), CF₃ and C₁₋₂alkoxy (e.g. methoxy), with the proviso that when R⁵ is H R⁶ is not H.

In another aspect of the invention one of R⁵ and R⁶ is H.

In another aspect of the invention R⁷ and R⁸ are independently selected from H or methyl. In another aspect R⁷ and R⁸ are both H.

In another aspect of the invention A is selected from the group consisting of

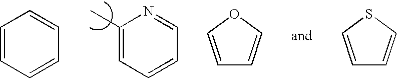

and A is unsubstituted or substituted by one or two R⁹.

In another aspect of the invention A is selected from the group consisting of

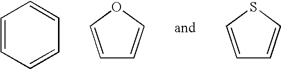

and A is unsubstituted or substituted by one or two R⁹.

In another aspect of the invention R⁹ is selected from the group consisting of hydroxy, halogen, C₁₋₃alkyl, C₁₋₂alkyl substituted by one to five fluorine atoms and C₁₋₃alkoxy.

Representative examples of R⁹ include methyl, methoxy, fluorine, bromine and hydroxy.

In another aspect of the invention R¹⁰ is selected from the group consisting of C₁₋₆alkyl (e.g. ethyl), phenyl and aminomethyl.

In another aspect of the invention B is

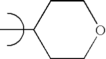

In another aspect of the invention n is 1 to 4.

In another aspect of the invention n is 0 to 2 (e.g. 1).

It is to be understood that the invention covers all combinations of particular aspects of the invention as described hereinabove.

Since the compounds of the present invention, in particular compounds of formula (I), are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compound of formula (I) may be used for preparing the more pure forms used in pharmaceutical compositions. Although the purity of intermediate compounds of the present invention is less critical, it will be readily understood that the substantially pure form is preferred as for the compounds of formula (I). Preferably, whenever possible, the compounds of the present invention are available in crystalline form.

When some of the compounds of this invention are allowed to crystallise or are recrysallised from organic solvents, solvent of recrystallisation may be present in the crystalline product. This invention includes within its scope such solvates. Similarly, some of the compounds of this invention may be crystallised or recrystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all the polymorphic forms of the compounds of formula (I).

Compounds of the invention are potent and selective inhibitors of COX-2. This activity is illustrated by their ability to selectively inhibit COX-2 over COX-1.

In view of their selective COX-2 inhibitory activity, the compounds of the present invention are of interest for use in human and veterinary medicine, particularly in the treatment of the pain (both chronic and acute), fever and inflammation of a variety of conditions and diseases mediated by selective inhibition of COX-2. Such conditions and diseases are well known in the art and include rheumatic fever; symptoms associated with influenza or other viral infections, such as the common cold; lower back and neck pain; headache; toothache; sprains and strains; myositis; sympathetically maintained pain; synovitis; arthritis, including rheumatoid arthritis; degenerative joint diseases, including osteoarthritis; gout and ankylosing spondylitis; tendinitis; bursitis; skin related conditions, such as psoriasis, eczema, burns and dermatitis; injuries, such as sports injuries and those arising from surgical and dental procedures.

The compounds of the invention are also useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; neuralgia, such as post-herpetic neuralgia and trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of the invention are also useful for the treatment of other conditions mediated by selective inhibition of COX-2.

For example, the compounds of the invention inhibit cellular and neoplastic transformation and metastatic tumour growth and hence are useful in the treatment of certain cancerous diseases, such as colonic cancer and prostate cancer. The compounds of the invention are also useful in reducing the number of adenomatous colorectal polyps and thus reduce the risk of developing colon cancer. The compounds of the invention are also useful in the treatment of cancer associated with overexpression of HER-2/neu, in particular breast cancer.

Compounds of the invention also prevent neuronal injury by inhibiting the generation of neuronal free radicals (and hence oxidative stress) and therefore are of use in the treatment of stroke; epilepsy; and epileptic seizures (including grand mal, petit mal, myoclonic epilepsy and partial seizures).

Compounds of the invention also inhibit prostanoid-induced smooth muscle contraction and hence are of use in the treatment of dysmenorrhoea and premature labour.

Compounds of the invention are also useful in the treatment of liver disease, such as inflammatory liver disease, for example chronic viral hepatitis B, chronic viral hepatitis C, alcoholic liver injury, primary biliary cirrhosis, autoimmune hepatitis, nonalcoholic steatohepatitis and liver transplant rejection.

Compounds of the invention inhibit inflammatory processes and therefore are of use in the treatment of asthma, allergic rhinitis and respiratory distress syndrome; gastrointestinal conditions such as inflammatory bowel disease, Crohn's disease, gastritis, irritable bowel syndrome and ulcerative colitis; and the inflammation in such diseases as vascular disease, migraine, periarteritis nodosa, thyroiditis, aplastic anaemia, Hodgkin's disease, sclerodoma, type I diabetes, myasthenia gravis, multiple sclerosis, sorcoidosis, nephrotic syndrome, Bechet's syndrome, polymyositis, gingivitis, conjunctivitis and myocardial ischemia.

Compounds of the invention are also useful in the treatment of ophthalmic diseases such as retinitis, retinopathies, uveitis and of acute injury to the eye tissue.

Compounds of the invention are also useful for the treatment of cognitive disorders such as dementia, particularly degenerative dementia (including senile dementia, Alzheimer's disease, Pick's disease, Huntington's chorea, Parkinson's disease and Creutzfeldt-Jakob disease), and vascular dementia (including multi-infarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment.

Compounds of the invention are also useful in the treatment of disorders ameliorated by a gastroprokinetic agent. Disorders ameliorated by gastroprokinetic agents include ileus, for example post-operative ileus and ileus during sepsis; gastroesophageal reflux disease (GORD, or its synonym GERD); gastroparesis, such as diabetic gastroparesis; and other functional bowel disorders, such as non-ulcerative dyspepsia (NUD) and non-cardiac chest pain (NCCP).

According to a further aspect of the invention, we provide a compound of formula (I) or a pharmaceutically acceptable salt thereof for use in human or veterinary medicine.

According to another aspect of the invention, we provide a compound of formula (I) or a pharmaceutically acceptable salt thereof for use in the treatment of a condition which is mediated by COX-2.

According to a further aspect of the invention, we provide a method of treating a human or animal subject suffering from a condition which is mediated by COX-2 which comprises administering to said subject an effective amount of a compound of formula (I) or a pharmaceutically acceptable salt.

According to a further aspect of the invention, we provide a method of treating a human or animal subject suffering from an inflammatory disorder, which method comprises administering to said subject an effective amount of a compound of formula (I) or a pharmaceutically acceptable salt thereof.

According to another aspect of the invention, we provide the use of a compound of formula (I) or a pharmaceutically acceptable salt thereof for the manufacture of a therapeutic agent for the treatment of a condition which is mediated by COX-2.

According to another aspect of the invention, we provide the use of a compound of formula (I) or a pharmaceutically acceptable salt thereof for the manufacture of a therapeutic agent for the treatment of an inflammatory disorder.

It is to be understood that reference to treatment includes both treatment of established symptoms and prophylactic treatment, unless explicitly stated otherwise.

It will be appreciated that the compounds of the invention may advantageously be used in conjunction with one or more other therapeutic agents. Examples of suitable agents for adjunctive therapy include a 5HT₁ agonist, such as a triptan (e.g. sumatriptan or naratriptan); an adenosine A1 agonist; an EP ligand; an NMDA modulator, such as a glycine antagonist; a sodium channel blocker (e.g. lamotrigine); a substance P antagonist (e.g. an NK₁ antagonist); a cannabinoid; acetaminophen or phenacetin; a 5-lipoxygenase inhibitor; a leukotriene receptor antagonist; a DMARD (e.g. methotrexate); gabapentin and related compounds; a tricyclic antidepressant (e.g. amitryptilline); a neurone stabilising antiepileptic drug; a mono-aminergic uptake inhibitor (e.g. venlafaxine); a matrix metalloproteinase inhibitor; a nitric oxide synthase (NOS) inhibitor, such as an iNOS or an nNOS inhibitor; an inhibitor of the release, or action, of tumour necrosis factor α; an antibody therapy, such as a monoclonal antibody therapy; an antiviral agent, such as a nucleoside inhibitor (e.g. lamivudine) or an immune system modulator (e.g. interferon); an opioid analgesic; a local anaesthetic; a stimulant, including caffeine; an H₂-antagonist (e.g. ranitidine); a proton pump inhibitor (e.g. omeprazole); an antacid (e.g. aluminium or magnesium hydroxide; an antiflatulent (e.g. simethicone); a decongestant (e.g. phenylephrine, phenylpropanolamine, pseudoephedrine, oxymetazoline, epinephrine, naphazoline, xylometazoline, propylhexedrine, or levo-desoxyephedrine); an antitussive (e.g. codeine, hydrocodone, carmiphen, carbetapentane, or dextramethorphan); a diuretic; or a sedating or non-sedating antihistamine. It is to be understood that the present invention covers the use of a compound of formula (I) or a pharmaceutically acceptable salt thereof in combination with one or more other therapeutic agents.

The compounds of formula (I) and their pharmaceutically acceptable salts are conveniently administered in the form of pharmaceutical compositions. Thus, in another aspect of the invention, we provide a pharmaceutical composition comprising a compound of formula (I) or a pharmaceutically acceptable salt thereof adapted for use in human or veterinary medicine. Such compositions may conveniently be presented for use in conventional manner in admixture with one or more physiologically acceptable carriers or excipients.

The compounds of formula (I) and their pharmaceutically acceptable salts may be formulated for administration in any suitable manner. They may, for example, be formulated for topical administration or administration by inhalation or, more preferably, for oral, transdermal or parenteral administration. The pharmaceutical composition may be in a form such that it can effect controlled release of the compounds of formula (I) and their pharmaceutically acceptable salts.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative.

Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

As stated above, the compounds of the invention may also be used in combination with other therapeutic agents. The invention thus provides, in a further aspect, a combination comprising a compound of formula (I) or a pharmaceutically acceptable salt thereof together with a further therapeutic agent.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable salt thereof is used in combination with a second therapeutic agent active against the same disease state the dose of each compound may differ from that when the compound is used alone. Appropriate doses will be readily appreciated by those skilled in the art.

A proposed daily dosage of a compound of formula (I) for the treatment of man is 0.01 mg/kg to 500 mg/kg, such as 0.05 mg/kg to 100 mg/kg, e.g. 0.1 mg/kg to 50 mg/kg, which may be conveniently administered in 1 to 4 doses. The precise dose employed will depend on the age and condition of the patient and on the route of administration. Thus, for example, a daily dose of 0.25 mg/kg to 10 mg/kg may be suitable for systemic administration.

Compounds of formula (I) and pharmaceutically acceptable salts thereof may be prepared by any method known in the art for the preparation of compounds of analogous structure.

Compounds of formula (I) and pharmaceutically acceptable salts thereof may be prepared by a process which comprises:

reacting an amine R¹R²NH of formula (II) or a protected derivative thereof with a compound of formula (III)

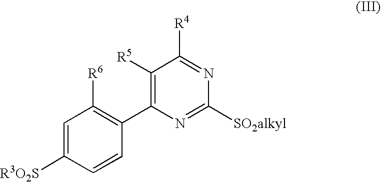

and thereafter and if necessary,

interconverting a compound of formula (I) into another compound of formula (I); and/or

deprotecting a protected derivative of compound of formula (I).

The overall synthesis of a compound of formula (I) is shown in Schemes 1 to 3 below.

In Scheme 1, R¹, R², R⁴, R⁵ and R⁶ are as defined in formula (I) above, R³ is C₁₋₆alkyl; alkyl is a straight or branched chain alkyl group, for example methyl, ethyl, n-propyl, i-propyl, n-butyl, s-butyl or t-butyl group.

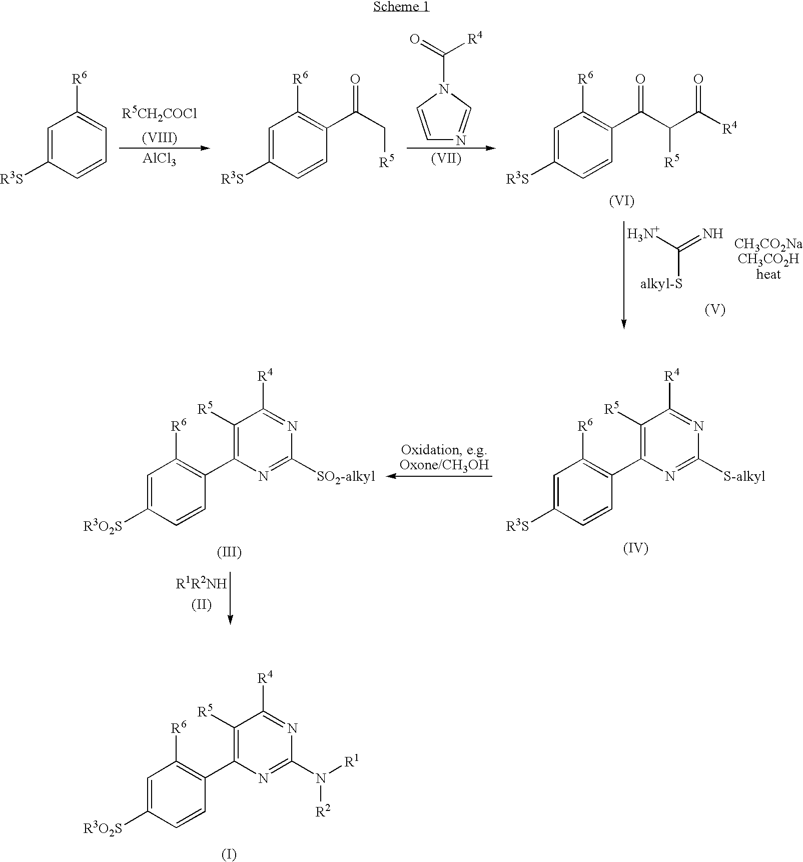

In Scheme 2, R¹, R², R⁴ and R⁶ are as defined in formula (I) above, R³ is C₁₋₆alkyl, and R⁵ is C₁₋₄alkyl or C₃₋₁₀cycloalkylC₀₋₆alkyl; alkyl is a straight or branched chain alkyl group, for example methyl, ethyl, n-propyl, i-propyl, n-butyl, s-butyl or t-butyl group.

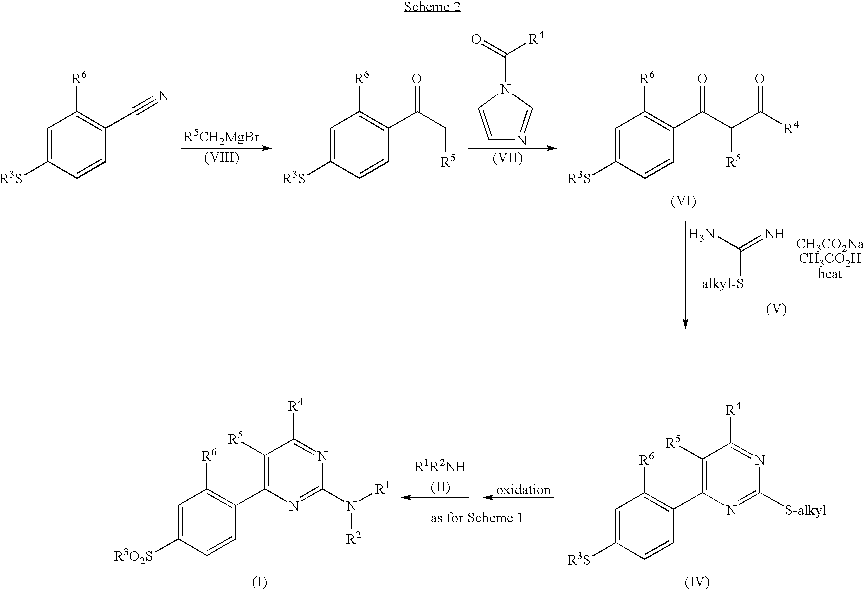

In Scheme 3, R¹, R², R⁴ and R⁵ are as defined in formula (I) above, R³ is C₁₋₆alkyl, and R⁶ is halogen or C₁₋₄alkoxy; alkyl is a straight or branched chain alkyl group, for example methyl, ethyl, n-propyl, i-propyl, n-butyl, s-butyl or t-butyl group; X is a suitable leaving group such as halogen; and CDI is 1,1′-carbonyldiimidazole.

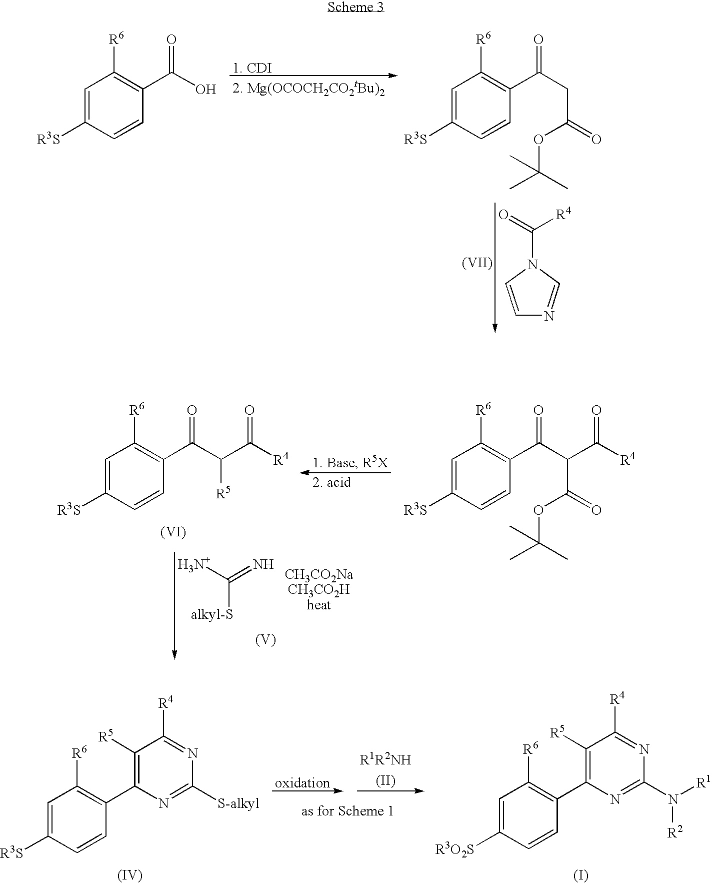

Referring to Schemes 1, 2 and 3, the treatment of compounds of formula (III) with an amine of formula (II) is conveniently carried out in a solvent, such as nitrile (e.g. methylnitrile) and at elevated temperature (e.g. from about 50° C. to reflux). An excess of the amine may be used in place of the solvent.

Alternatively, the treatment of compounds of formula (III) with an amine of formula (II) is conveniently carried out in a solvent, such as a tertiary amine (e.g. NMP), and at between ambient and elevated temperature (e.g. ambient temperature). Use of, for example, NMP as solvent has the advantage that after completion of the reaction the desired compound of formula (I) may be precipitated from the reaction mixture by the addition of water, allowing for easier isolation and purification.

Conveniently the oxidation shown in Schemes 1, 2 and 3 is effected using a monopersulfate compound, such as potassium peroxymonosulfate (known as Oxone™) and the reaction is carried out in a solvent, such as an aqueous alcohol, (e.g. aqueous methanol), and at between −78° C. and ambient temperature.

Alternatively, the oxidation shown in Schemes 1, 2 and 3 may be effected using hydrogen peroxide in the presence of catalytic sodium tungstate dihydrate. The reaction may be carried out in a solvent such as acetic acid and at between ambient temperature and reflux (e.g. 50° C.).

Referring to Schemes 1, 2 and 3, the cyclisation of diones of formula (VI) to give the corresponding pyrimidines of formula (IV) is conveniently carried out employing a thioronium salt such as a 2-methyl-2-thiopseudourea sulfate and under reflux.

Referring to Scheme 1, acid chlorides of formula (VIII) are either known compounds or may be prepared by conventional chemistry.

Compounds of formula (VII) in Schemes 1, 2 and 3 are either known compounds or may be prepared by conventional chemistry.

It will be appreciated by those skilled in the art that certain of the procedures described in Schemes 1, 2 and 3 for the preparation of compounds of formula (I) or intermediates thereto may not be applicable to some of the possible substituents.

It will be further appreciated by those skilled in the art that it may be necessary or desirable to carry out the transformations described in Schemes 1, 2 and 3 in a different order from that described, or to modify one or more of the transformations, to provide the desired compound of formula (I).

In one variation of Schemes 1, 2 and 3, compounds of formula (III) wherein R³ is C₁₋₆alkyl or NH₂ may be prepared by oxidising a compound of formula (IV)A:

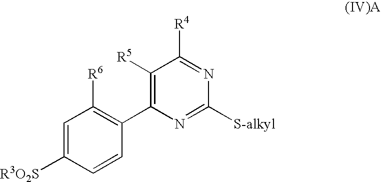

under oxidation conditions described hereinabove. Compounds of formula (IV)A may be prepared according to the general procedures of Schemes 1, 2 and 3 by employing sulphonyl derivatives in place of the corresponding sulfide compounds.

It will be appreciated by those skilled in the art that compounds of formula (I) may be prepared by interconversion, utilising other compounds of formula (I) as precursors. Suitable interconversions, such as alkylations, are well known to those skilled in the art and are described in many standard organic chemistry texts, such as ‘Advanced Organic Chemistry’ by Jerry March, fourth edition (Wiley, 1992), incorporated herein by reference. For example, compounds of formula (I) wherein R¹ is C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkane, (CR⁴R⁵)ₙA (with the proviso that n is not zero) and (CR⁴R⁵)ₙB may be prepared by alkylating the corresponding compound of formula (I) wherein R¹ is H.

Acylation of compounds of formula (I) wherein R³ is NH₂, to provide corresponding acylated benzenesulphonamide derivatives, may be carried out by conventional means, for example by employing conventional acylating agents such as those described in ‘Advanced Organic Chemistry’, pp 417-424, incorporated herein by reference.

As will be appreciated by those skilled in the art it may be necessary or desirable at any stage in the synthesis of compounds of formula (I) to protect one or more sensitive groups in the molecule so as to prevent undesirable side reactions.

The protecting groups used in the preparation of compounds of formula (I) may be used in conventional manner. See, for example, those described in ‘Protective Groups in Organic Synthesis’ by Theodora W Green and Peter G M Wuts, second edition, (John Wiley and Sons, 1991), incorporated herein by reference, which also describes methods for the removal of such groups.

Amines of formula (II) are either known compounds or may be prepared by literature methods, such as those described in ‘Comprehensive Organic Transformations: a guide to functional group preparations’ by Richard Larock (VCH, 1989), incorporated herein by reference.

Thioronium salts of formula (V) are either known compounds or may be prepared by literature methods, such as those described in A H Owens et al, Eur J Med Chem, 1988, 23(3), 295-300, incorporated herein by reference.

Certain intermediates described above are novel compounds, and it is to be understood that all novel intermediates herein form further aspects of the present invention. Compounds of formulae (III) and (IV) are key intermediates and represent a particular aspect of the present invention.

Conveniently, compounds of the invention are isolated following work-up in the form of the free base. Pharmaceutically acceptable acid addition salts of the compounds of the invention may be prepared using conventional means.

Solvates (e.g. hydrates) of a compound of the invention may be formed during the work-up procedure of one of the aforementioned process steps.

The Intermediates and Examples which follow illustrate the invention but do not limit the invention in any way. All temperatures are in ° C.

### Intermediate 1

### 1-[4-(Methylthio)phenyl]propan-1-one

A solution of 4-(methylthio)benzonitrile (7.46 g, 50 mmol) in anhydrous tetrahydrofuran (50 mL) was added dropwise (over a period of 0.5 hours) to a solution of ethyl magnesium bromide (100 mL, 1.0 M solution in tetrahydrofuran) with stirring, under a nitrogen atmosphere. The resultant reaction mixture was stirred for 2 hours, before being heated to reflux for a period of 5 hours. The reaction mixture was then stirred overnight at ambient room temperature.

The reaction mixture was cooled in an ice-bath to a temperature of 0° C. before 2M sulphuric acid (15 mL) was added dropwise (the temperature being maintained below 20° C.). The resultant thick yellow suspension was filtered under vacuo and the resultant filter cake was washed generously with ethyl acetate. The filtrate containing the product was then washed with 2M sulphuric acid, followed by saturated sodium hydrogen carbonate and then finally with water. The organic phase was separated and dried over anhydrous magnesium. The magnesium sulphate was filtered off under suction and the filtrate was evaporated under vacuo to yield the desired product as a solid (8.3 g, 92%). C₁₀H₁₂SO (Calculated 180.2). Found MH+ 181.

### Intermediate 2

### 4,4,4-Trifluoro-2-methyl-1-[4-(methylthio)phenyl]butane-1,3-dione

A solution of 1-[4-(methylthio)phenyl]propan-1-one (4.0 g, 22 mmol) in anhydrous tetrahydrofuran (50 mL) was cooled to a temperature of −78° C. under a nitrogen atmosphere. To this solution was added gradually a solution of sodium bis(trimethylsilylamide) (24 mL, 24 mmol, 1.0M solution in tetrahydrofuran) whilst maintaining the temperature at −78° C. On completion of the addition of the sodium bis(trimethylsilylamide) the resultant reaction mixture was stirred for a further 45 minutes at a temperature of −78° C., before the reaction mixture was allowed to warm to a temperature of −20° C. (briefly). The reaction mixture was then cooled to a temperature of −78° C. To the resultant sodio-salt of 1-[4-(methylthio)phenyl]propan-1-one was added a solution of 1-(trifluoroacetyl) imidazole (2.8 mL, 25 mmol) in anhydrous tetrahydrofuran (10 mL). On completion of the addition of the solution of 1-(trifluoroacetyl) imidazole, the reaction mixture was stirred for a further 5 minutes, before the reaction mixture was allowed to warm to ambient room temperature. The reaction mixture was then stirred overnight at ambient room temperature. The reaction mixture was carefully quenched with water and acidified with 1M hydrochloric acid. The reaction mixture was then extracted into ethyl acetate. The organic phase was separated and combined before being washed with 1 M hydrochloric acid, followed by water. The organic phase was separated and dried over anhydrous magnesium sulphate. The magnesium sulphate was then filtered off under suction and the filtrate evaporated to yield the crude product as an oil. The crude product was purified via silica gel chromatography eluting with ethyl acetate:cyclohexane (1:19). This gave the desired product as a colourless solid (2.3 g, 38%).

C₁₂H₁₁O₂F₃S (Calculated 276.3). Found MH+ 277.

### Intermediate 3

### 5-Methyl-2-(methylthio)-4-[4-(methylthio)phenyl]-6-(trifluoromethyl)pyrimidine

A mixture of 4,4,4-trifluoro-2-methyl-1-[4-(methylthio)phenyl]butane-1,3-dione (2.2 g, 8.0 mmol), 2-methyl-2-thiopseudourea sulphate (2.7 g, 9.6 mmol) and anhydrous sodium acetate (1.3 g, 16 mmol) in glacial acetic acid (10 mL) was heated to reflux, overnight, under a nitrogen atmosphere. The reaction mixture was diluted with water and the product was extracted into ethyl acetate. The organic phase was separated and dried over anhydrous magnesium sulphate. The magnesium sulphate was filtered off under suction and the filtrate was then evaporated under vacuo to give the crude product. The product was purified via silica gel chromatography, eluting with cyclohexane. The desired product was obtained as a colourless solid (2.2 g, 83%).

C₁₄H₁₃N₂F₃S₂ (Calculated 330.4). Found MH+ 331.

### Intermediate 4

### 5-Methyl-2-(methylsulfonyl)-4-[4-(methylsulphonyl)phenyl]-6-(trifluoromethyl)pyrimidine

To a solution of 5-methyl-2-(methylthio)-4-[4-(methylthio)phenyl]-6-(trifluoromethyl)pyrimidine (2.2 g, 6.6 mmol) in methanol (150 mL) was added potassium peroxymonosulphate (OXONE) (18 g, 29 mmol) followed by water (30 mL). The resultant reaction mixture was stirred overnight (under a nitrogen atmosphere) at ambient room temperature. The reaction mixture was filtered under suction to remove suspended colourless solid and the filtrate was then evaporated under vacuo to give the product. The product was dissolved in dichloromethane and dried by passing the dichloromethane solution through a hydrophobic frit. The dichloromethane was removed by evaporation under vacuo to yield the desired product as a crystalline colourless solid (2.6 g, 100%).

C₁₄H₁₃F₃N₂O₄S₂ (Calculated 394.4). Found MH+ 395 (MNH4+ 412).

### EXAMPLE 1

### N-benzyl-5-methyl-4-[4-(methylsulfonyl)phenyl]-6-(trifluoromethyl)pyrimidin-2-amine

To a solution of 5-methyl-2-(methylsulfonyl)-4-[4-(methylsulphonyl)phenyl]-6-(trifluoromethyl)pyrimidine (20 mg, 0.051 mmol) in 1-methyl-2-pyrrolidinone (2.0 mL) was added benzylamine (28 μL, 0.25 mmol). The resultant solution was stirred overnight at ambient room temperature. The reaction mixture was diluted with water (5 mL) before being applied to a reverse phase column (SPE cartridge C 18 Octadecyl, 10 g, 60 mL) eluting with water (initially) to remove 1-methyl-2-pyrrolidinone, followed by acetonitrile to elute the crude product. The product was purified by silica gel chromatography eluting with dichloromethane, followed by ethyl acetate:cyclohexane (1:1 v/v). This afforded the title compound as a colourless oil that solidified to a colourless solid on standing (12.8 mg, 59.9%).

C₂₀H₁₈F₃N₃O₂S (Calculated 421.4). Found MH+=422.

### EXAMPLE 2

### N-isobutyl-5-methyl-4-[4-(methylsulfonyl)phenyl]-6-(trifluoromethyl)pyrimidin-2-amine

To a solution of 5-methyl-2-(methylsulfonyl)-4-[4-(methylsulphonyl)phenyl]-6-(trifluoromethyl)pyrimidine (20 mg, 0.051 mmol) in acetonitrile (5.0 mL) was added isobutylamine (25 μL, 0.25 mmol). The resultant solution was heated overnight at a temperature of 80° C. The reaction mixture was allowed to cool before the solvent was removed by evaporation under vacuo. The resultant oil was purified by silica gel chromatography (SPE cartridge SiO₂, 10 g, 60 mL) eluting with dichloromethane, followed by ethyl acetate:cyclohexane (1:1 v/v). This afforded the title compound as a colourless solid (15.6 mg, 79%). C₁₇H₂₀F₃N₃O₂S (Calculated 387.4). Found MH+=388.

Examples 3 to 15 as shown in Table 1 that follows, were prepared in the manner described for Examples 1 and 2.
an open bracket, (, is used to mark the bond between the substituent and the nitrogen atom to which it is attached.

TABLE 1

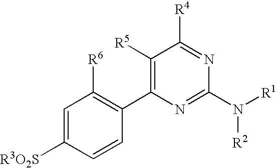

| | | | | | | | MS |
|---|---|---|---|---|---|---|---|
| Ex | R¹ | R² | R³ | R⁴ | R⁵ | R⁶ | (MH+) |
| 3 | H | 2-hydroxybenzyl | CH₃ | CF₃ | CH₃ | H | 438 |
| 4 | H | 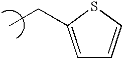 | CH₃ | CF₃ | CH₃ | H | 428 |
| 5 | H | 4-methylbenzyl | CH₃ | CF₃ | CH₃ | H | 436 |
| 6 | H | cyclohexyl | CH₃ | CF₃ | CH₃ | H | 414 |
| 7 | H | 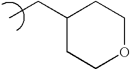 | CH₃ | CF₃ | CH₃ | H | 430 |
| 8 | H | n-propyl | CH₃ | CF₃ | CH₃ | H | 374 |
| 9 | H | 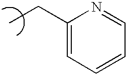 | CH₃ | CF₃ | CH₃ | H | 423 |
| 10 | H | 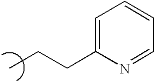 | CH₃ | CF₃ | CH₃ | H | 437 |
| 11 | H | n-butyl | CH₃ | CF₃ | CH₃ | H | 388 |
| 12 | H | 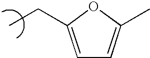 | CH₃ | CF₃ | CH₃ | H | 426 |
| 13 | H | 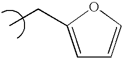 | CH₃ | CF₃ | CH₃ | H | 412 |
| 14 | H | 4-methoxybenzyl | CH₃ | CF₃ | CH₃ | H | 452 |
| 15 | H | 4-fluorobenzyl | CH₃ | CF₃ | CH₃ | H | 440 |

Biological Data

Inhibitory activity against microsomal h-COX2 was assessed against a microsomal preparation from baculovirus infected SF9 cells. An aliquot of microsomal preparation was thawed slowly on ice and a 1/40,000 dilution prepared from it into the assay buffer (sterile water, degassed with argon containing 100 mM HEPES (pH 7.4), 10 mM EDTA (pH7.4), 1 mM phenol, 1 mM reduced glutathione, 20 mg/ml gelatin and 0.001 mM Hematin). Once diluted the enzyme solution was then sonicated for 5 seconds (Branson sonicator, setting 4, 1 cm tip) to ensure a homogeneous suspension. 155 μl enzyme solution was then added to each well of a 96-well microtitre plate containing either 5 μl test compound (40× required test concentration) or 5 μl DMSO for controls. Plates were then mixed and incubated at room temperature for 1 hour. Following the incubation period, 40 μl of 0.5 μM arachidonic acid was added to each well to give a final concentration of 0.1 μM. Plates were then mixed and incubated for exactly 10 minutes (room temperature) prior to addition of 25 μl 1M HCl (hydrochloric acid) to each well to stop the reaction. 25 μl of 1M NaOH (sodium hydroxide) was then added to each well to neutralise the solution prior to determination of PGE₂ levels by enzyme immunoassay (EIA). Results are expressed as % inhibition of PGE2 release by the compound compared to solvent control at 10 micromolar concentration.

| Example No. | COX-2% inhibition | COX-2 IC50 (μM) |
|---|---|---|
| 1 | 61 | 1.3 |
| 2 | | 6.6 |
| 3 | | 2.5 |
| 4 | | 0.56 |
| 5 | | 15.3 |
| 6 | | 2.3 |
| 7 | | >100 |
| 8 | | 26.7 |
| 9 | | >98.6 |
| 10 | | 71.2 |
| 11 | 43 | 6.3 |
| 12 | 11 | |
| 13 | 43 | |
| 14 | 23 | |
| 15 | 35 | |

1. A compound of formula (I) 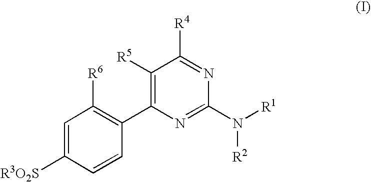
or a pharmaceutically acceptable salt thereof, in which:
R¹ and R² are independently selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁷R⁸)ₙ and B(CR⁷R⁸)ₙ;
R³ is selected from the group consisting of C₁₋₆alkyl, NH₂ and R¹⁰CONH;
R⁴ is C₁₋₂alkyl substituted by one to five fluorine atoms;
R⁵ is selected from the group consisting of H, C₁₋₄alkyl, C₁₋₂alkyl substituted with one to five fluorine atoms, halogen and C₃₋₁₀cycloalkylC₀₋₆alkyl, with the proviso that when R⁶ is H R⁵ is not H.
R⁶ is selected from the group consisting of H, C₁₋₄alkyl, C₁₋₂alkyl substituted with one to five fluorine atoms, halogen, C₁₋₄alkoxy, CN, NO₂, C₁₋₆alkylOCO, NH₂CO, C₁₋₆alkylNHCO, NH₂, C₁₋₆alkylNH, (C₁₋₆alkyl)₂N, (C₁₋₆alkyl)₂NCO, C₁₋₆alkylCONH, NH₂SO₂, C₁₋₆alkylNHSO₂, (C₁₋₆alkyl)₂NSO₂, C₁₋₆alkylSO₂NH, ArSO₂NH, C₁₋₆alkylSO₂, ArSO₂, C₃₋₁₀cycloalkylC₀₋₆alkyl, C₃₋₆alkenyl and C₃₋₆alkynyl, with the proviso that when R⁵ is H R⁶ is not H.
R⁷ and R⁸ are independently selected from H or C₁₋₆alkyl;
A is an unsubstituted 5- or 6-membered heteroaryl or an unsubstituted 6-membered aryl, or a 5- or 6-membered heteroaryl or a 6-membered aryl substituted by one or more R⁹;
R⁹ is selected from the group consisting of hydroxy, halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
R¹⁰ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂C₁₋₆alkyl, C₁₋₆alkylOCONHC₁₋₆alkyl and C₁₋₆alkylCONHC₁₋₆alkyl;
B is selected from the group consisting of 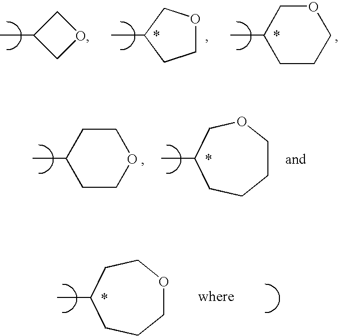
defines the point of attachment of the ring; and
n is 0 to 4.

2. The compound as claimed in claim 1 wherein
R¹ and R² are independently selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁷R⁸)ₙ and B(CR⁷R⁸)ₙ;
R³ is selected from the group consisting of C₁₋₆alkyl, NH₂ and R¹⁰CONH;
R⁴ is C₁₋₂alkyl substituted by one to five fluorine atoms;
R⁵ is selected from the group consisting of H, C₁₋₄alkyl, C₁₋₂alkyl substituted with one to five fluorine atoms and C₃₋₁₀cycloalkylC₀₋₆alkyl, with the proviso that when R⁶ is H R⁵ is not H.
R⁶ is selected from the group consisting of H, C₁₋₄alkyl, C₁₋₂alkyl substituted with one to five fluorine atoms, halogen and C₁₋₄alkoxy, with the proviso that when R⁶ is H R⁵ is not H.
R⁷ and R⁸ are independently selected from H or C₁₋₆alkyl;
A is an unsubstituted 5- or 6-membered heteroaryl or an unsubstituted 6-membered aryl, or a 5- or 6-membered heteroaryl or a 6-membered aryl substituted by one or more R⁹;
R⁹ is selected from the group consisting of halogen, C₁₋₆alkyl, C₁₋₆alkyl substituted by one or more fluorine atoms, C₁₋₆alkoxy, C₁₋₆alkoxy substituted by one or more F, NH₂ SO₂ and C₁₋₆alkyl SO₂;
R¹⁰ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂C₁₋₆alkyl, C₁₋₆alkylOCONHC₁₋₆alkyl and C₁₋₆alkylCONHC₁₋₆alkyl;
B is selected from the group consisting of 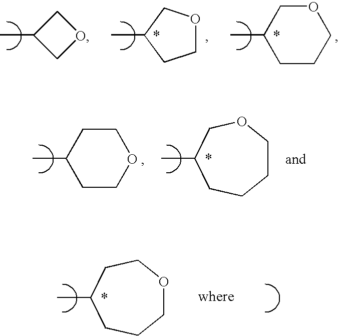
defines the point of attachment of the ring; and
n is 0 to 4.

3. The compound as claimed in claim 1 wherein R¹ is H.

4. The compound as claimed in claim 1 wherein R² is selected from the group consisting of C₁₋₆alkyl, C₃₋₁₀cycloalkylC₀₋₆alkyl (such as C₃₋₁₀cycloalkyl or C₃₋₇cycloalkylmethyl), A(CR⁷R⁸)ₙ and B(CR⁷R⁸)ₙ.

5. The compound as claimed in claim 1 wherein R³ is C₁₋₆alkyl.

6. The compound as claimed in claim 1 wherein R⁴ is CHF₂, CH₂F or CF₃.

7. The compound as claimed in claim 1 wherein R⁵ is H or C₁₋₄alkyl, with the proviso that when R⁶ is H R⁵ is not H.

8. The compound as claimed in claim 1 wherein R⁶ is selected from the group consisting of H, C₁₋₂alkyl (e.g. methyl), CF₃ and C₁₋₂alkoxy (e.g. methoxy), with the proviso that when R⁵ is H R⁶ is not H.

9. The compound as claimed in claim 1 wherein R⁷ and R⁸ are independently selected from H or methyl.

10. The compound as claimed in claim 1 wherein A is selected from the group consisting of 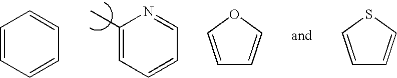
and A is unsubstituted or substituted by one or two R⁹.

11. The compound as claimed in claim 1 wherein R⁹ is selected from the group consisting of hydroxy, halogen, C₁₋₃alkyl, C₁₋₂alkyl substituted by one to five fluorine atoms and C₁₋₃alkoxy.

12. The compound as claimed in claim 1 wherein R¹⁰ is selected from the group consisting of C₁₋₆alkyl (e.g. ethyl), phenyl and aminomethyl.

13. The compound as claimed in claim 1 wherein B is

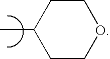

14. The compound as claimed in claim 1 wherein n is 0 to 2.

15. A process for the preparation of a compound as defined in claim 1, which comprises:
(A), reacting an amine HNR¹R² of formula (II) or a protected derivative thereof with a compound of formula (III) 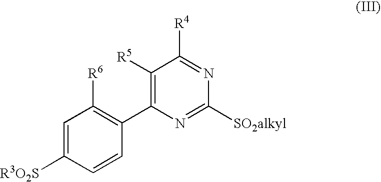
and thereafter and if necessary,
(B), interconverting a compound of formula (I) into another compound of formula (I); and/or
(C), deprotecting a protected derivative of compound of formula (I).

16. A pharmaceutical composition comprising a compound as defined in claim 1 in admixture with one or more physiologically acceptable carriers or excipients.

17. (canceled)

18. A method of treating a subject suffering from a condition which is mediated by COX-2 which comprises administering to said subject an effective amount of a compound as defined in claim 1.

19. A method of treating a subject suffering from an inflammatory disorder, which method comprises administering to said subject an effective amount of a compound as defined in claim 1.

20-21. (canceled)

22. The method according to claim 18, wherein said subject is a human.

23. The method according to claim 19, wherein said subject is a human.
